(19) United States
(12) Patent Application Publication
(10) Pub. No.: US 2020/0282182 A1
(43) Pub. Date: Sep. 10, 2020

(54) CATHETER SECURING SYSTEM AND METHODS OF USE

(71) Applicants: Gurvan Edmond Blackman, Robbinsville, NJ (US); David Christopher Feldstein, Haddonfield, NJ (US)

(72) Inventors: Gurvan Edmond Blackman, Robbinsville, NJ (US); David Christopher Feldstein, Haddonfield, NJ (US)

(21) Appl. No.: 16/809,258

(22) Filed: Mar. 4, 2020

Related U.S. Application Data
(60) Provisional application No. 62/813,230, filed on Mar. 4, 2019.

(51) Int. Cl. A61M 25/02

(52) U.S. Cl. CPC A61M 25/02; A61M 2025/0266

(57) ABSTRACT

A catheter securing device includes a housing having an upper arm, a lower arm and a hinge disposed between the upper arm and the lower arm and capable of transitioning the housing between a closed condition and an open condition, the upper arm having a recess, an adhesive layer disposed on the lower arm, a first adapter configured to be at least partially disposed within the housing, the first adapter having an upper member, a lower member and a hinge disposed between the upper member and the lower member and capable of transitioning the first adapter between a closed condition and an open condition, the lower member defining a lower concavity, and the upper member defining an upper concavity, the lower concavity and the upper concavity collectively defining a first lumen sized to accept a portion of a catheter.

### CROSS-REFERENCE TO RELATED APPLICATIONS

This application claims priority to U.S. Provisional Application Ser. No. 62/813,230, filed Mar. 4, 2019, entitled “CATHETER SECURING SYSTEM AND METHODS OF USE,” the contents of which are fully incorporated as if fully set forth herein.

### FIELD OF THE DISCLOSURE

The present disclosure relates to catheter securing systems for use in medical settings. More particularly the present disclosure relates to devices and methods to aid in securing a catheter to a patient's body and prevent accidental removal.

### BACKGROUND OF THE DISCLOSURE

Medical procedures may require the use of devices for intravenous access for prolonged periods of time. In some cases, percutaneous catheters may be used to access the patient's vasculature. Examples of such catheters may include central venous catheters (i.e., central lines), peripherally inserted central catheters (PICCs), midlines, and dialysis/apheresis catheters.

In some cases, a substantial portion of the catheter will hang loosely from a patient's body. This makes the catheter prone to breakage, damage, displacement and inadvertent removal when a portion of the device becomes hooked on the patient's clothing or bedding, or on other medical devices. One common technique to secure a catheter is to secure a portion of it via sutures. This is uncomfortable for the patient, time-consuming for the physician and altogether unreliable.

### SUMMARY OF THE DISCLOSURE

In some embodiments, a catheter securing device includes a housing having an upper arm, a lower arm and a hinge disposed between the upper arm and the lower arm and capable of transitioning the housing between a closed condition and an open condition, the upper arm having a recess, an adhesive layer disposed on the lower arm, a first adapter configured to be at least partially disposed within the housing, the first adapter having an upper member, a lower member and a hinge disposed between the upper member and the lower member and capable of transitioning the first adapter between a closed condition and an open condition, the lower member defining a lower concavity, and the upper member defining an upper concavity, the lower concavity and the upper concavity collectively defining a first lumen sized to accept a portion of a catheter.

### BRIEF DESCRIPTION OF THE DISCLOSURE

Various embodiments of the presently disclosed catheter securing devices are shown herein with reference to the drawings, wherein:

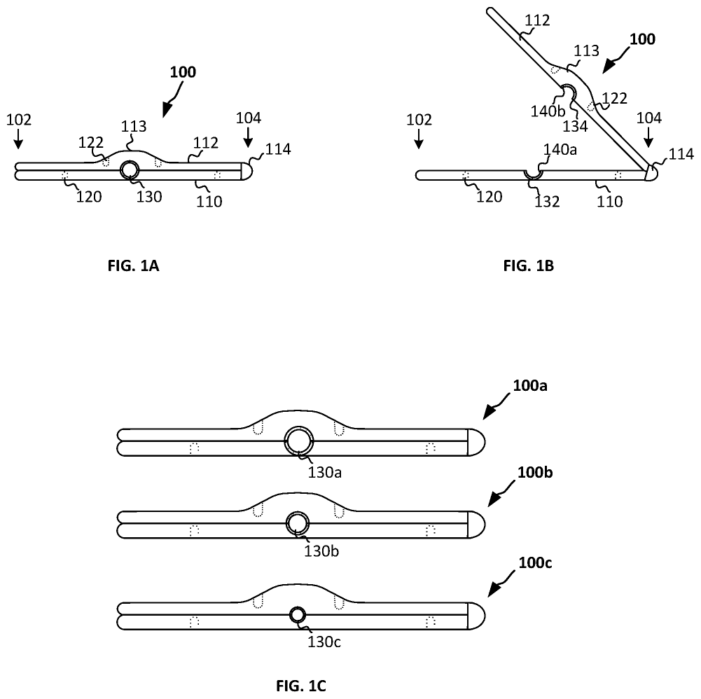

FIG. 1A is a schematic front view of an adapter of a catheter securing device in the closed condition according to one embodiment of the present disclosure;

FIG. 1B is a schematic front view of the adapters of FIG. 1A in an open condition;

FIG. 1C are schematic front views of adapter similar to those of FIG. 1A having differently-sized lumens;

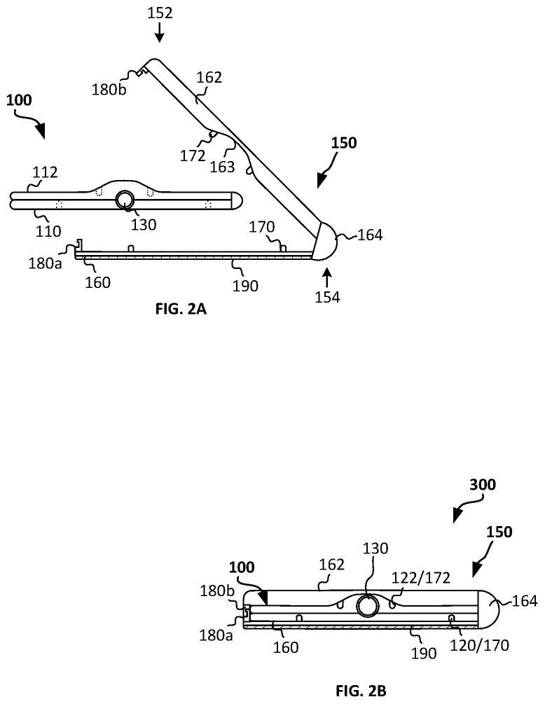

FIG. 2A is a schematic front view of a housing of a catheter securing device in the open condition capable of accepting the adapter of FIG. 1A according to one embodiment of the present disclosure;

FIG. 2B is a schematic front view of the catheter securing device including the adapter and the housing in a closed condition;

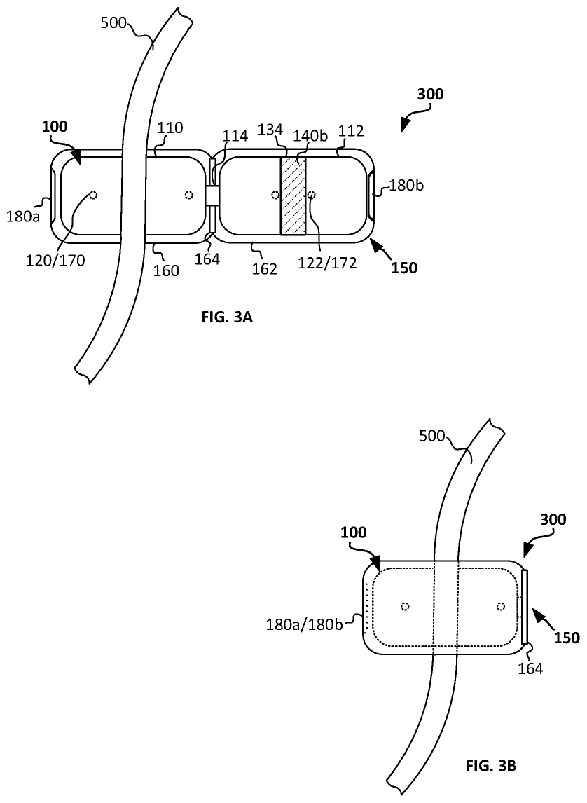

FIGS. 3A-B are schematic top views of the catheter securing device of FIGS. 1A-2B being used to secure a catheter in the open and closed conditions;

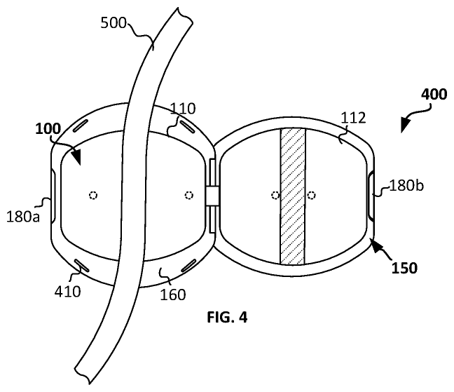

FIG. 4 is a schematic top view of another catheter securing device of being used to secure a catheter.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to catheter securing devices and their methods of use, conventional devices suffer from some shortcomings as described above.

There therefore is a need for further improvements to the devices, systems, and methods of securing and stabilizing catheters, such as catheters that are at least partially placed within the body. These catheters may include abscess drainage catheters, chest tubes, nephrostomy tubes, biliary drainage catheters, cholecystostomy tubes, or other percutaneous drainage catheters. Among other advantages, the present disclosure may address one or more of these needs.

The present disclosure relates to a catheter securing system that lowers the possibility of displacement or accidental removal from a patient. The catheter securing system may also improve patient comfort and allow for rapid exchange of catheters within the system. A catheter securing system according to the present disclosure includes two main portions in the form of an adapter and a housing capable of accepting the adapter.

FIG. 1A shows one example of an adapter 100 according to the present disclosure. Adapter 100 extends between proximal end 102 and distal end 104, and includes a lower member 110, an upper member 112 and a hinge 114 coupling the lower member to the upper member adjacent distal end 104. Hinge 114 allows the adapter to transition from a closed condition in which the upper and lower members are substantially parallel to an open condition in which the upper and lower members are angled relative to one another and spaced apart to accept a portion of a catheter. In some examples, the hinge 114 may allow the upper and the lower members to form a 180-degree angle in the open condition such that the two members are capable of laying completely flat next to one another with the hinge therebetween.

As shown, upper member 112 may include a raised center 113 to provide additional spacing for forming a lumen, although it will be understood that entire upper member may instead be flat. A pair of lower slots 120 are defined in lower member 110, which are openings defined in a bottom surface of the lower member sized to receive pins of the housing as will be described in greater detail with respect to the housing. Lower slots 120 may be spaced apart as shown so that they are disposed on either side of a lumen 130. A pair of upper slots 122 may be formed in upper member 112 and spaced from the lumen as shown. In at least some examples, slots 120,122 are formed of the same shape and size. Alternatively, slots 102 may be formed of different shapes and/or sizes than slots 122 as desired.

As shown in FIG. 1B, lower member 110 may define a curved concavity 132 on a surface opposite the slots 120. As shown, curved concavity 132 is shaped as a semi-circle although it will be understood that other shapes are possible. Upper member 112 may define an upper concavity 134 similar to lower concavity 132 positioned directly above the lower concavity 132 when the device is in the closed condition, the two concavities collectively defining a lumen 130 having a circular cross-section through the adapter in the closed condition. The size of lumen 130 may be varied as desired and may be sized to accept common diameters of catheters, the size being fractionally smaller than a given catheter to ensure a secure fit. For example, lumen 130 may be configured to accept catheters of sizes, for example, from 4 French to 30 French. In some examples, the lumen is configured to accept catheters larger than 30 French. The lumen may be large enough to accommodate the catheter within impinging on the lumen of the catheter, but small enough to properly secure the catheter and prevent it from sliding back and forth through the lumen. An adhesive 140a,140b may be applied to the inside surface of concavities 132,134, the adhesive being strong enough to secure a portion of a catheter within the lumen and prevent the catheter from moving relative to the adapter.

Suitable materials for the adapter may include a polymeric material, such as silicone, thermoplastic polyurethanes (TPU), rubber, metal, plastic, polypropylene, polyethylene, acrylonitrile butadiene styrene (ABS), high impact polystyrene (HIPS), polyvinyl chloride (PVC) polycarbonate, thermoplastic elastomers, polybutylene terephthalate, ethylene vinyl acetate, nylon a low-density polyethylene, linear low-density polyethylene, and suitable combinations thereof. Alternatively, adapter 100 may also be formed at least partially or entirely of a metal such a stainless steel, titanium, or other biocompatible metal.

As shown in FIG. 1C, a plurality of adapters 100a,100b,100c may be formed, each having the same-sized lower and upper members, and the same configuration of slots 120,122 so that the adapter are all interchangeable and coupleable to the same housing. The only difference between the adapters 100,100b,100c are the sizes of the concavities defined by the upper and lower members that form the lumen for accepting differently-sized catheters. Specifically, the length, width and height of adapters 100a,100b,100c are all the same, and the location of the slots 120,122 are also the same. In some examples, the raised center 113 provides a larger area into which the concavities will be defined to accommodate catheters of a larger size.

FIGS. 2A-B illustrate a housing 150 that may be used with adapters 100. Housing 150 extends between a proximal end 152, and a distal end 154, and includes a hinge 164 adjacent the distal end that couples a lower arm 160 to an upper arm 162, the upper arm having a recess 163 tailored to match the curvature of raised center 113 of adapter 100. Hinge 164 may allow the housing to transition between a closed condition and an open condition similar to the hinge of adapter 100. In some cases, hinge 164 may allow the upper and lower arms of the housing to open a full 180 degrees such that the two arms are capable of laying completely flat next to one another with the hinge therebetween as shown, for example, in FIGS. 3A-B.

Housing 150 may be formed of the same materials as adapter 100. In other examples, the adapter and the housing are formed of different materials. Housing may further have a low profile so that, even when an adapter is disposed therein, the housing has a profile of ¼ inches or less, ½ inches or less, or ¾ inches or less.

Housing 150 further includes a pair of lower pins 170 and a pair of upper pins 172, the lower and upper pins being sized, spaced and capable of mating with slots 120,122 of the adapter, which receive the pins therein. Housing 150 further includes a lower clasp 180a, and an upper clasp 180b that mate to lock the housing in the closed condition and secure the adapter therein.

Coupled to lower arm 160 on a surface furthest from the upper arm is an adhesive layer 190 configured to attached the housing to the patient's skin. Suitable materials for adhesive layer 190 may include adhesives, glues and materials such as acrylate, including methacrylates and/or epoxy diacrylates.

FIG. 2B illustrates a housing 150 that is in the closed position, with an adapter 100 nested therein. As shown, pins 170,172 are coupled to slots 120,122, and recess 163 receives raised center 113 therein. Clasps 180a,180b are also coupled together to lock the housing in the closed condition and prevent the housing or adapter from being opened.

In use, a catheter securing device 300 including the adapter 100 and the housing 150 may be placed in the open configuration (FIG. 3A), and the housing may be coupled to the patient's skin at a predetermined location via adhesive layer 190. Lower arm 160 of housing 150 may be coupled to lower member 110 of adapter 100. A portion of a catheter 500 may be placed in the lower concavity of adapter 100. Upper member 112 of adapter 100 may be coupled to upper arm 162 of housing 150 as shown in FIG. 3A, and both the upper member and the upper arm may be rotated over their respective hinges to close the device by bringing the upper member 112 over the lower member 110 and the upper and lower concavities together to form the lumen. It will be noted that in some examples, adapter 100 may be transitioned to the closed condition, and housing 150 may be closed thereafter (e.g., the two portions may be closed sequentially instead of being closed together). Clasps 180a,180b of housing 150 may mate with one another to secure the catheter within lumen 130. Additionally, adhesive material 140a,140b surrounding the catheter may prevent the catheter 500 from sliding relative to the catheter securing device 300 so that with the housing being secured to the patient's body, and the catheter secured within the lumen of the adapter, the distal end of catheter that is within the body is affixed at a spaced distance with respect to the catheter securing device 300 and the device is incapable of being inadvertently pushed further into the body or out of the body. If a physician or caretaker decides to exchange a catheter or use the securing system for a different purpose, another adapter may be interchanged with the first adapter, the second adapter having an appropriate lumen size for securing the new catheter. In this manner, catheters of different sizes may be secured to the same device, and the housing may be coupled to the patient once without having to secure it via sutures or otherwise cause discomfort to the patient.

It will be understood that the shape and/or size of the adapter and/or housing may be varied as needed. For example, FIG. 4 illustrates a housing/adapter combination each having a clam-shaped perimeter of the upper and lower portions. Other shapes are possible such as generally circular, oval, square-shaped, triangular or diamond-shaped upper and lower portions of the housing and/or adapters. Additionally, in addition to or instead of the adhesive materials, eyelets 410 may be formed in, for example, the lower arm of the housing to help secure the housing to the patient's skin. As shown, the lower arm of the housing of device 400 is oversized in comparison to the upper arm so that the eyelets are exposed when the device is in the closed position. Various sizes, shapes of the eyelets, and two or more eyelets may be disposed at other positions on the housing, instead of or in addition to the adhesive.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A catheter securing device comprising:
a housing having an upper arm, a lower arm and a hinge disposed between the upper arm and the lower arm and capable of transitioning the housing between a closed condition and an open condition, the upper arm having a recess;
an adhesive layer disposed on the lower arm;
a first adapter configured to be at least partially disposed within the housing, the first adapter having an upper member, a lower member and a hinge disposed between the upper member and the lower member and capable of transitioning the first adapter between a closed condition and an open condition, the lower member defining a lower concavity, and the upper member defining an upper concavity, the lower concavity and the upper concavity collectively defining a first lumen sized to accept a portion of a catheter; and
an adhesive material covering at least one of the upper concavity and the lower concavity.

2. The catheter securing device of claim 1, wherein the first adapter further comprises at least one upper slot and at least one lower slot defined within the upper member, and the lower member, respectively.

3. The catheter securing device of claim 2, wherein the housing further comprises at least one upper pin, and at least one lower pin disposed on the upper arm and the lower arm, respectively, the at least one upper pin and the at least one lower pin being configured and arranged to mate with the at least one upper slot and the at least one lower slot, respectively.

4. The catheter securing device of claim 1, wherein the housing further comprises a first clasp on the upper arm, and a second clasp on the lower arm, the first clasp and the second clasp being capable of mating to lock the housing in the closed condition.

5. The catheter securing device of claim 1, wherein the first adapter is configured and arranged to be fully covered by the housing in the closed condition.

6. The catheter securing device of claim 1, further comprising a second adapter having a same length, width and height as the first adapter, the second adapter defining a second lumen that is larger than the first lumen.

7. The catheter securing device of claim 1, wherein the first lumen is configured to accept a catheter having a diameter of 4 French.

8. A kit for securing a catheter, comprising:
a housing having an upper arm, a lower arm and a hinge disposed between the upper arm and the lower arm and capable of transitioning the housing between a closed condition and an open condition, the upper arm having a recess;
an adhesive layer disposed on the lower arm;
a first adapter configured to be at least partially disposed within the housing, the first adapter having a first upper member, a first lower member and a first hinge disposed between the first upper member and the first lower member and capable of transitioning the first adapter between a closed condition and an open condition, the first lower member and the first upper member collectively defining a first lumen sized to accept a portion of a catheter; and
a second adapter interchangeable with the first adapter within the housing, the second adapter having a second upper member, a second lower member and a second hinge disposed between the second upper member and the second lower member and capable of transitioning the second adapter between a closed condition and an open condition, the second lower member and the second upper member collectively defining a second lumen that is larger than the first lumen of the first adapter.

9. The kit of claim 8, wherein the first adapter and the second adapter have a same length, a same width and a same height.

10. The kit of claim 8, wherein the first upper member and the first lower member are capable of forming a 180-degree angle in the open condition.

11. The kit of claim 8, further comprising a third adapter having a third lumen that is larger than the first lumen and the second lumen.

12. The kit of claim 11, wherein the third adapter is interchangeable with the first adapter.

13. The kit of claim 8, wherein the first adapter further comprises at least one upper slot and at least one lower slot defined within the upper member, and the lower member, respectively.

14. The kit of claim 13, wherein the housing further comprises at least one upper pin, and at least one lower pin disposed on the upper arm and the lower arm, respectively, the at least one upper pin and the at least one lower pin being configured and arranged to mate with the at least one upper slot and the at least one lower slot.

15. A method of securing a catheter comprising:
providing a housing having an upper arm, a lower arm and a hinge disposed between the upper arm and the lower arm and capable of transitioning the housing between a closed condition and an open condition, the housing being coupled to an adhesive layer;
inserting a first adapter within the housing, the first adapter defining a first lumen sized to accept a first portion of a first catheter, and having an adhesive material surrounding the lumen; and
replacing the first adapter with a second adapter, the second adapter defining a second lumen sized to accept a first portion of a second catheter, and having an adhesive material surrounding the lumen.

16. The method of claim 15, further comprising the step of placing the housing in a closed condition to lock one of the first adapter and the second adapter therein.
